(19) United States
(12) Patent Application Publication
(10) Pub. No.: US 2025/0041167 A1
(43) Pub. Date: Feb. 6, 2025

(54) System and Method for Darkening Skin in Selected Areas on and Around the Face, Neck and Body

(71) Applicant: Benjamin P. Caughlin, Chicago, IL (US)

(72) Inventor: Benjamin P. Caughlin, Chicago, IL (US)

(21) Appl. No.: 18/365,164

(22) Filed: Aug. 3, 2023

(51) Int. Cl. A61K 8/02; A61K 8/35; A61Q 19/04

(52) U.S. Cl. CPC A61K 8/0212; A61K 8/35; A61Q 19/04

(57) ABSTRACT

A system and method for selectively darkening specific areas of the face, neck, and body. A mask is provided that retains a volume of a skin darkening compound. Masks of different shapes and sizes are provided. A neck mask is designed to reduce the prominence of the neck by adding the visual illusion of shading and contour to the skin of the neck. A cheek mask is provided that darkens the cheeks and provides the illusion of shading and contour on the cheeks. A nose mask is provided that darkens and deemphasizes the nose while leaving the central bridge open to create thinness and contour. A body mask is provided that darkens and contours selected areas of the chest and abdomen. The masks adhere to selected regions for a period of time. The skin darkening compound contained within the masks darkens the skin in specific areas and along precise lines. The darkening provides a distinct shaded base for the later application of cosmetics. The darkening is natural, water proof and can be layered upon. Natural body contours, freckles and nevi will still be visible.

### BACKGROUND OF THE INVENTION

### 1. Field of the Invention

In general, the present invention relates to face, neck, and body masks that are used to apply various skin darkening compounds to the skin for prolonged periods of time. More particularly, the present invention relates to the shapes of masks and how those shapes and the darkening compound affect the areas of application.

### 2. Prior Art Description

There are many types of compounds that individuals apply to the skin of the face, neck, and body. These compounds include various cleaners, moisturizers, wrinkle serums and the like. Typically, such compounds are applied directly to the skin of the face. However, such compounds are also used to line masks that are applied to the face. Masks are commonly used to retain a compound on the skin for a prolonged period of time. Masks prevent the applied compound from being touched and wiped away. Accordingly, masks are commonly used while a person sleeps to prevent pillow contact. Many masks are used to cover the entire face. Such masks are typically used with skin cleansers or skin moisturizers. Such full masks are exemplified by U.S. Patent Application Publication No. 2008/0014231 to Okano.

Some compounds, such as anti-wrinkle serums, can be expensive and are intended to be applied sparingly to only targeted areas of the face and neck. If such compounds are used with a mask, the mask is usually small or segmented so it can apply the compound to only targeted areas of the face and neck. Such prior art masks are exemplified by U.S. Patent Application Publication No. 2019/0298038 to Miles, U.S. Patent Application Publication No. 2009/0280150 to Kamen, and U.S. Pat. No. RE38,246 to Leonard. Since such prior art masks show compounds being applied to the skin that to the skin that have no significant effect upon the color of the skin, such prior art masks tend to have broadly curved edges and a size that significantly overlaps the needed area of coverage.

Although masks are often used to apply various compounds to the skin, masks are rarely ever used to increase color and/or darkening of the skin. Cosmetics are traditionally compounds used to apply contour by increasing the color and darkening appearance to the skin. Cosmetics must be applied with precision to the face and neck, wherein different shades of color are applied to different areas of the face and neck. Often the colors are blended and shaded to emphasize or deemphasize different facial features. Furthermore, cosmetics are typically applied in layers wherein color layers are applied atop a foundation layer. The application of cosmetics can therefore be considered a precise art that must be practiced differently on different people. Accordingly, cosmetics cannot just be applied to a mask that is applied to the face. Such a mask would lack the precision and color complexities required to artfully apply cosmetics.

Although masks cannot be used to artfully apply cosmetics, it has been discovered that masks can be used to apply base shading to the skin prior to the application of other cosmetics. The present invention is an improved mask system that can apply shaping to the face, neck, and body in a precise manner, therein providing a depth of darkening, color, contour, and contrast to the skin that is difficult to achieve using cosmetics alone. In addition, the skin darkening created by the present invention produces will be a natural water proof staining of the skin. The darkening is natural, water proof and can be layered upon. Natural body contours, freckles and nevi will still be visible. The improved mask system and methodology are described and claimed below.

### SUMMARY OF THE INVENTION

The present invention is a system and method for selectively contouring via a darkening of specific areas of the face, neck, and body. A mask is provided that retains a volume of a skin darkening compound. The skin darkening compound naturally stains the skin and is not a cosmetic. It is waterproof and only removable by abrasion and removal of the outer most layer of skin. The skin darkening compound stains the skin in areas of contact between the mask and the skin. Masks of different shapes and sizes are provided. A neck mask is shaped to contact the neck under the mandible between the sternocleidomastoid muscles on either side including the submental area. The neck mask has a periphery made from straight lines, that produces clear lines of color transition on the skin. The darkening is designed to reduce the prominence of the neck by adding the visual illusion of darkening and concavity shading to the skin of the neck.

A cheek mask is provided that is applied to the face below the cheek bones and above the mandible body. The cheek mask darkens the cheek skin and provides the illusion of shading on the cheeks that is similar to a concavity in this area. This makes the cheeks seem slightly withdrawn and thins the appearance of the face.

A nose mask is provided that covers the sides of the nose and the areas below the brows of the eyes near the nose. This darkening deemphasizes the nose, by keeping the midline of the bridge light and the sides darkened. This mimics how cosmetics are used to contour the nose and to make it appear sharper and thinner, therein making the face seem more delicate.

Other masks, such as chest or abdomen contour masks can also be used to selectively darken sections of the chest to emphasize or deemphasize various areas and muscle groups. All, some, or any one of the masks adhere to selected regions of the face, neck, and body for a period of time. The skin darkening compound contained within the masks darkens the skin in specific areas and along precise lines. The darkening provides a distinct shaded base for the later application of cosmetics.

### BRIEF DESCRIPTION OF THE DRAWINGS

For a better understanding of the present invention, reference is made to the following description of exemplary embodiments thereof, considered in conjunction with the accompanying drawings, in which:

FIG. 1 shows a head darkened in specific areas of the face and neck;

FIG. 2 shows the head of FIG. 1 in conjunction with different masks that produce the specific areas of darkening on the face and neck;

FIG. 3 is a cross-section of a segment of mask shown in circle 3 of FIG. 2;

FIG. 4 shows a front view of an exemplary neck mask;

FIG. 5 shows the neck mask of FIG. 4 applied to a face;

FIG. 6 is a front view of an exemplary cheek mask;

FIG. 7 shows the cheek mask applied to a face;

FIG. 8 is a front view of an exemplary nose mask;

FIG. 9 shows the nose mask applied to a face; and

FIG. 10 shows a body mask shown in conjunction with a body's torso.

### DETAILED DESCRIPTION OF THE DRAWINGS

Although the present invention system and methodology can be embodied in many ways, only a few exemplary embodiments are illustrated. The exemplary embodiments are being shown for the purposes of explanation and description. The exemplary embodiments are selected in order to set forth some of the best modes contemplated for the invention. The illustrated embodiments, however, are merely exemplary and should not be considered as limitations when interpreting the scope of the appended claims.

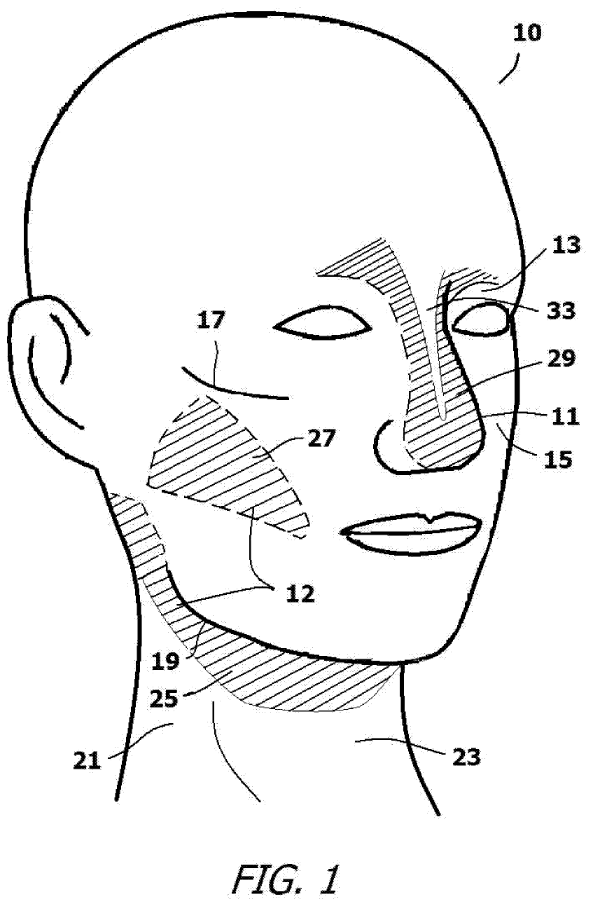

Referring to FIG. 1, a head 10 is shown containing selected areas 12 that are intentionally darkened in order to obtain various aesthetic effects. The head 10 includes the anatomical reference points of a nose 11, eyebrows 13, cheeks 15, cheek bones 17, a ramus and body of mandible 19, sternocleidomastoid muscle, 21 and a neck 23. The selected areas 12 illustrated as being darkened include a first neck area 25 under the mandible 19 and between the sternocleidomastoid muscles 21 including inferior and posterior to the ramus and body of the mandible 21 and the submental area under the mandible 21. The selected areas 12 also include a cheek area 27 on the cheeks 15 below the cheek bones 17 and the bridge 33 and nasal side wall areas 29 of the nose 11. Skin darkening is used to deemphasize the selected areas 12 and create the illusion of a concavity. A darkened of the nasal side walls 28 in relation to the midline bridge 33 on a nose 11 can make a nose 11 seem more slender and thinner than it actually is. A darkened cheek area 27 can make a face seen thinner than it actually is in a manner similar to buccal fat pad removal. Lastly, a darkened neck area 25 can deemphasize fullness, double chins, and jowls, therein making the neck seen thinner and more contoured.

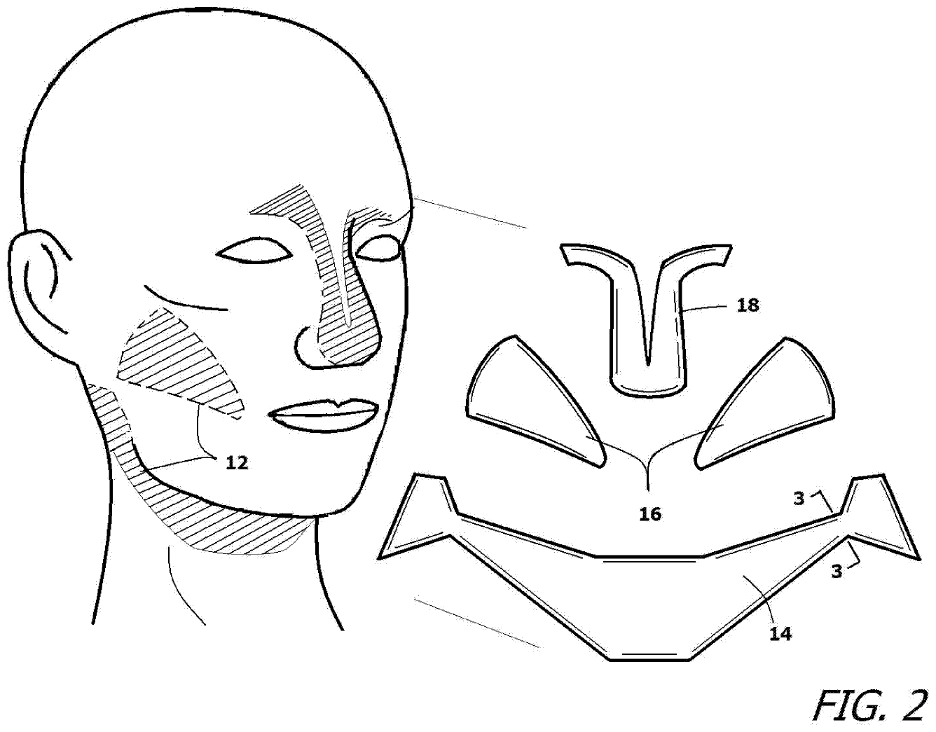

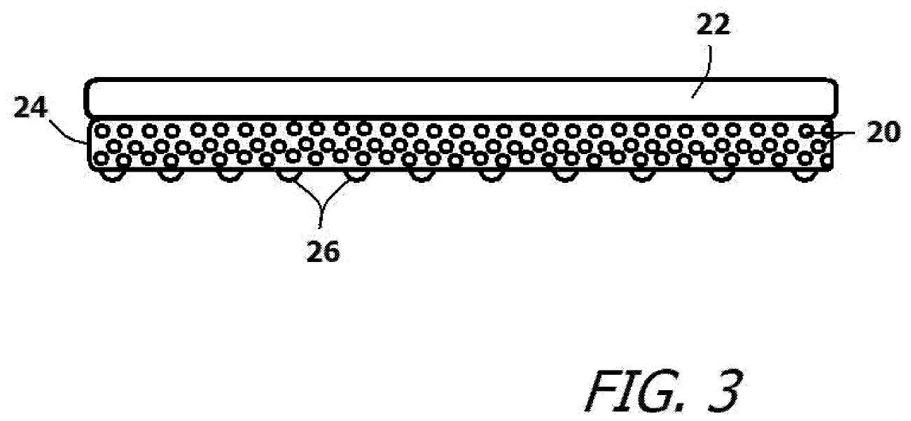

Referring to FIG. 2 and FIG. 3, in conjunction with FIG. 1, it can be seen that to make the selected areas 12 darker, applicator masks 14, 16, 18 are applied to the skin over the selected areas 12 that are to be darkened. Each of the applicator masks 14, 16, 18 holds a volume of a darkening skin staining compound 20. The darkening skin staining compound 20 preferably produces a darkened color that is similar to the color of the patient's natural melanin 20. The preferred skin staining compound 20 is a solution of dihydroxyacetone, which is also known as glyceone. Dihydroxyacetone is a colorless 3-carbon sugar that creates a chemical reaction with amino acids in the surface cells of the skin, therein producing a darkening effect by creating melanonids. This gives the skin a similar color to natural melanin stimulation. Dihydroxyacetone does not damage skin as it only affects the outermost cells of the epidermis. The dihydroxyacetone does not induce a tanning response. Rather, the dihydroxyacetone stains the surface layers of the skin by covalently binding to free amine groups in keratin proteins, followed by a polymerization reaction. Since the staining is limited to only the skin cells contacted by the dihydroxyacetone, the staining of the skin is temporary. The staining typically lasts up to seven days after an application. It is water, sweat and wear proof. Cosmetics and skincare products can be applied naturally on top of it for further contouring. It can only be removed by skin removal techniques such as derma-abrasion. Currently, dihydroxyacetone is the only active ingredient that is approved by the US Food and Drug Administration (FDA) for sunless tanning. The degree of skin darkening is related to the concentration of the dihydroxyacetone. The preferred concentrations are preferably between 2 percent and 10 percent by volume.

Each mask 14, 16, 18 is made of layers. A first substrate layer 22 is provided. The substrate layer 22 can be made from many traditional skin mask materials, such as a film of polypropylene, silicone, bio-cellulose or woven bamboo. Such films can be impervious to water and are also highly flexible. An absorptive layer 24 is bonded to the substrate layer 22. The absorptive layer 24 is saturated or partially saturated with a desired concentration of dihydroxyacetone. The concentration depends upon the natural pigmentation of the skin being affected and the degree of darkening desired during the period of application. In some instances, microdots of adhesive 26 are applied to the exposed surface of the absorptive layer 24, wherein the adhesive 26 binds readily to skin. In this manner, the masks 14, 16, 18 can adhere to the skin, therein exposing the dihydroxyacetone to the skin. In other cases, the viscosity of the solution is used to cause adherence.

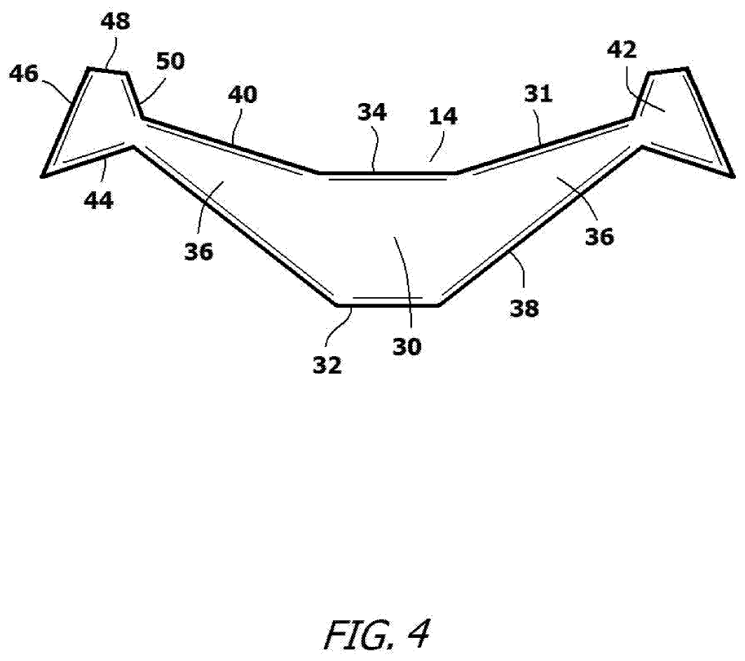

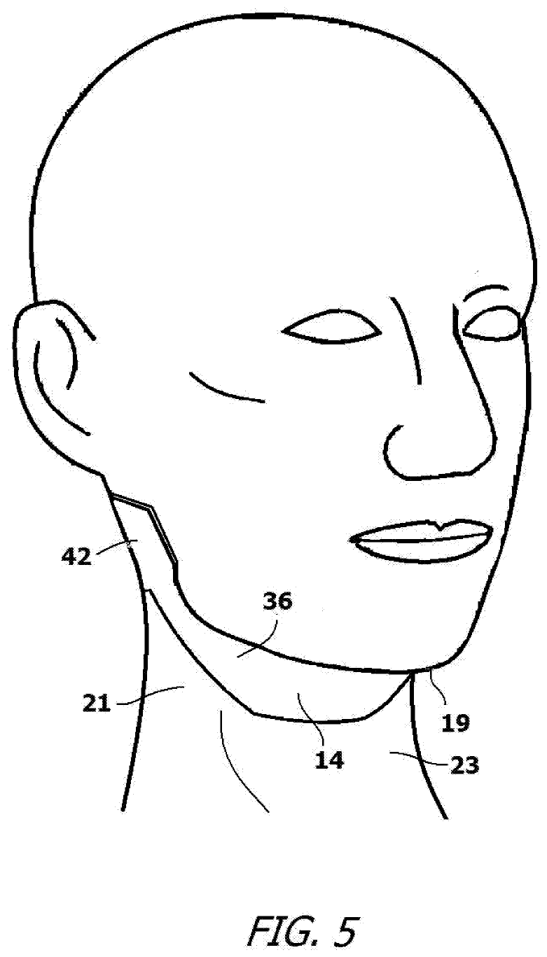

Referring to FIG. 4 and FIG. 5 in conjunction with FIG. 1 and FIG. 3, it can be seen that the neck mask 14 is designed to be applied to the neck 23 under the mandible 19. The neck mask 14 applies the skin darkening compound 20 to the neck area 25 along the neck 23 just under the mandible which optimizes the slimming contour concavity illusion. The neck mask 14 has a periphery 31 that is made from various straight and curved lines. Accordingly, when the neck mask 14 is applied to the neck area 25, the neck mask 14 will darken skin along distinct lines. The lines are positioned to create the optical illusion that certain regions in the neck area 25 are less prominent and concave than others.

The neck mask 14 is symmetrically formed around a center section 30. The center section 30 has a bottom edge 32 and a top edge 34 that are parallel or near parallel. The distance between the bottom edge 32 and the top edge 34 is preferably 50 mm+/−10 mm depending upon the size of the user's head 10.

Arms 36 extend away from the center section 30. The arms 36 taper as the arms 36 extend away from the center section 30. Each arm 36 has a lower edge 38 that extends from the bottom edge 32 of the center section 30 at an angle of between thirty degrees and sixty degrees. Each arm 36 also has an upper edge 40 that extends away from the top edge 34 of the center section 30 at a preferred angle of between five degrees and twenty-five degrees. The different angles of the upper edge 40 and the lower edge 38 of each arm 36 makes the arms 36 taper as they extend away from the center section 30. After extending a distance of between 50 mm and 70 mm from the center section 30, the arms 36 extend into enlarged pads 42.

Each enlarged pad 42 has a polygonal shape made from straight sides. The polygonal shape has a first side 44 that extends at a descending angle from the lower edge 38 of the arm 36. The first side 44 of the enlarged pad 42 is preferably between 30 mm and 45 mm. A second side 46 extends at an ascending angle from the first side 44. The second side 46 has a preferred length of between 45 mm and 55 mm. A third side 48 extends from the second side 46 at an orientation near or at the horizontal, depending upon the jaw anatomy of the user. A fourth side 50 extends from the third side 48 to the top edge 40 of the arm 36. The fourth side 50 is between 15 mm and 25 mm in length. All the measures can vary significantly and have the potential to be customizable to specific patients. Likely the range is +/−20 mm in any dimension.

During application, the enlarged pads 42 of the neck mask 14 are placed on the head 10 just below the mandible anterior to the sternocleidomastoid muscle 21 lateral to the ramus of the mandible 19 and inferior to the body of the mandible 19. In this position, the straight sides of the enlarged pad 42 creates shape shadow lines that look like natural shading and contour. Between the enlarged pads 42, the neck mask 14 is pressed against the skin of the neck 23 under the mandible 19. The darkening of the skin in contact with the neck mask 14 makes the contacted neck area 25 more contoured, less prominent, therein making a person's neck 23 seem more concave, hollow, and thinner.

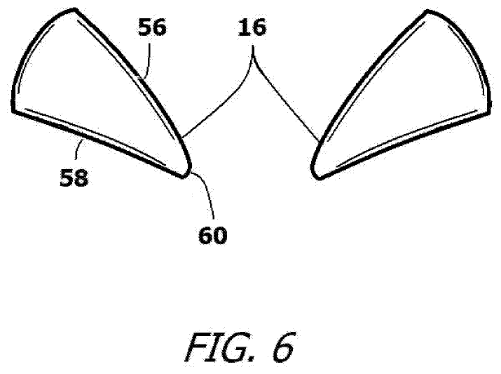

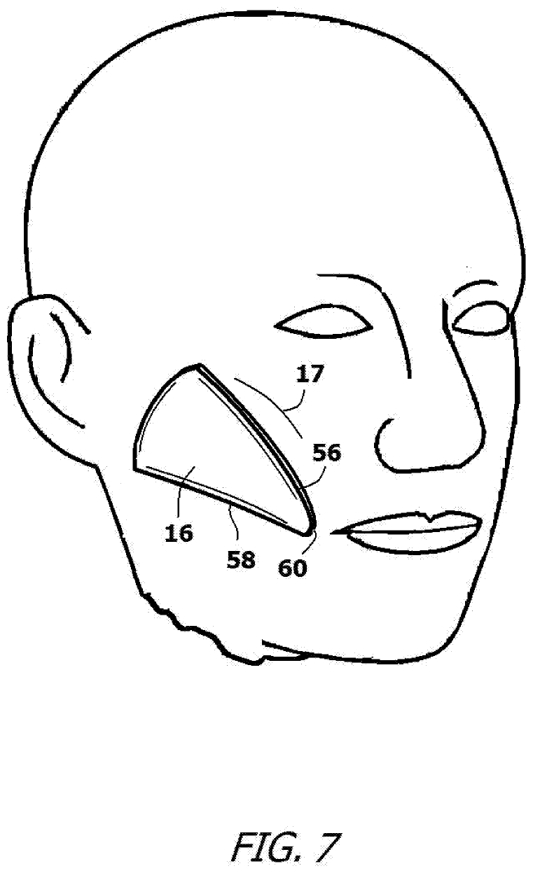

Referring to FIG. 6 and FIG. 7 in conjunction with FIG. 1 and FIG. 3, it can be seen that the cheek masks 16 are designed to be applied to the cheek areas 27 of the face just below the cheek bones 17 at the buccal space. The cheek mask 16 has a sloping surface 56 that passes under the cheek bone 17. The cheek mask 16 extends down from the sloping surface 56 to a jaw line edge 58. The jaw line edge 58 follows superior to the mandible 19 of the user. Both the top sloping surface 56 and the jaw line edge 58 converge toward a small, a common edge 60 that follows the corner of the mouth. The darkening of the skin in contact with the cheek mask 16 makes the cheek area 27 appear more contoured, more hollow, and less prominent under the cheek bones 17, therein making a person's face seem slimmer, contoured, and sharpened.

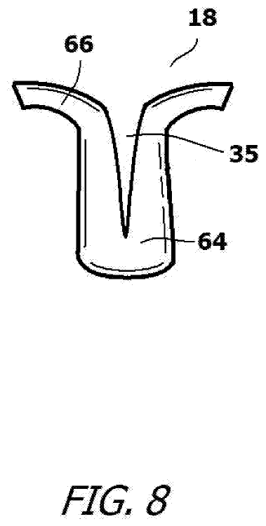

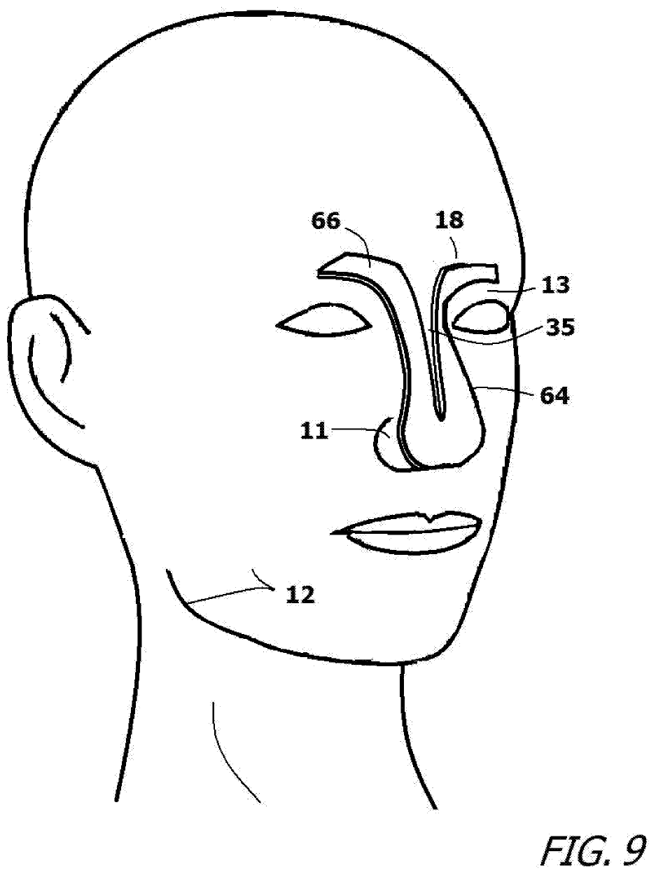

Referring to FIG. 8 and FIG. 9, it can be seen that the nose mask 18 is designed to create a narrowed appearance to the nose by being applied to the sidewall areas 29 of the nose 11 while keeping the central bridge area 33 open. The nose mask 18 has an elongated section 64 that is shaped to fit along the sidewall areas 29 of the nose 11. The nose mask 18 has a central slot 35 Two brow protrusions 66 extend from the top of the elongated section 64 to follow the brow tip aesthetic line. These brow protrusions 66 attach under the eyebrows 13. Accordingly, the lateral sidewall areas 29 of the nose 11 and the area under brow area 68 are darkened in relation to the central bridge and become less visually prominent. This darkening pattern is similar to how cosmetics are used to contour a nose.

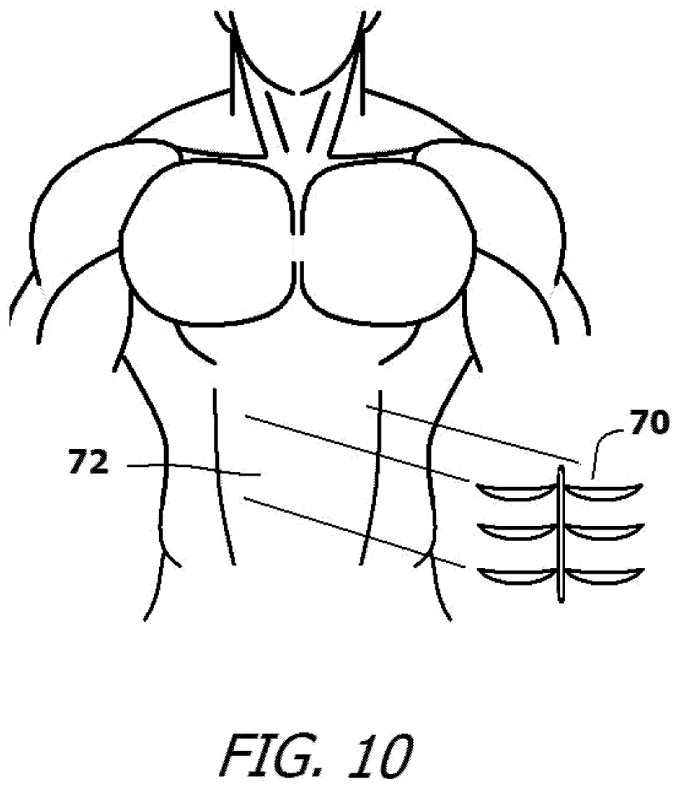

Referring to FIG. 10, an example of a body mask 70 is shown. In this embodiment the body mask 70 is used to darken and contour the areas 72 around the abdominal muscles. Body masks can also be used to darken skin on the torso to contour chest muscles on a man and breasts on a woman.

Referring to all figures, it will be understood that just one or all masks 14, 16, 18, 70 can be used to selectively darken areas of the face, neck, and body to make these areas less visually prominent and/or more contoured. The different masks 14, 16, 18, 70 can be applied to the skin for different lengths of time in order to achieve different degrees of darkening in different areas of application. The darkening of the skin cannot be washed away. The darkening of the skin can therefore be overlaid with tradition cosmetics and skin care products. This darkened areas can be made more, dak, less dark and/or be feathered into the natural color of the skin. It will be further understood that the embodiments of the present invention that are illustrated and described are merely exemplary and that a person skilled in the art can make many variations to those embodiments. For instance, the shape and size of the different masks 14, 16, 18, 70 can be customized to the features of the person using the masks 14, 16, 18, 70. All such embodiments are intended to be included within the scope of the present invention as defined by the appended claims.

What is claimed is:

1. A method of selectively darkening shaped areas of skin to create a cosmetic effect on said shaped areas, said method comprising:
providing at least one mask that is shaped to match said shaped areas, wherein said at least one mask contains a darkening compound that chemically darkens said skin when in contact with said skin;
applying said at least one mask to said shaped areas of said skin a period of time, wherein said darkening compound darkens said skin only in said shaped areas; and
removing said at least one mask from said skin.

2. The method according to claim 1, wherein said shaped areas are selected from a group comprising face areas, neck areas, chest areas and abdomen areas.

3. The method according to claim 1, wherein said skin darkening compound is a solution of dihydroxyacetone.

4. The method according to claim 3, wherein said solution of dihydroxyacetone is a solution containing between 2 percent and 10 percent dihydroxyacetone.

5. On a person having a head, with a mandible, sternocleidomastoid muscle, and a neck area that extends under the mandible, a method of selectively darkening at least some of said neck area, comprising:
providing a mask that contains a skin darkening compound, wherein said mask has a shape that enables said mask to conform to selected regions of said neck area;
adhering said mask to said selected regions of said neck area for a length of time, wherein said skin darkening compound darkens said selected regions of said neck area where said mask contacts said neck area.

6. The method according to claim 5, wherein said shape of said mask includes a central section and two tapered arms that symmetrically extend from opposite sides of said central section, wherein each of said tapered arms terminate with an enlarged pad.

7. The method according to claim 5, wherein adhering said mask to said neck area includes adhering said enlarged pads to said head under said mandible, anterior to said sternocleidomastoid muscle.

8. The method according to claim 6, wherein said center section has a top edge, a bottom edge and two sides, wherein said top edge is parallel to said bottom edge and said tapered arms extend from said sides of said central section.

9. The method according to claim 6, wherein each said enlarged pad has a polygonal perimeter.

10. The method according to claim 5, wherein said skin darkening compound is a solution of dihydroxyacetone.

11. On a person having a face, cheek bones, and a mandible, a method of selectively darkening the skin on the face in an area under a cheek bone, said method comprising:
providing a mask that contains a skin darkening compound, wherein said mask has a shape that enables said mask to conform to the face in an application area under the cheek bones;
adhering said mask to said application area for a period of time, wherein said skin darkening compound darkens said application area.

12. The method according to claim 11, wherein adhering said mask to said application area includes adhering said mask between the cheek bone and the mandible.

13. The method according to claim 11, wherein said mask has a sloping surface that passes under the cheek bone, and a jaw line edge that follows the mandible, wherein both the sloping surface and the jaw line edge converge toward a common edge.

14. The method according to claim 13, wherein said skin darkening compound is a solution of dihydroxyacetone.

15. On a person having a face, nose, a nose bridge, and eyebrows, a method of selectively darkening the skin on the face on and above the nose, said method comprising:
providing a mask that contains a skin darkening compound, wherein said mask is shaped to cover portions of the nose and areas under the eyebrows;
adhering said mask to an application area that covers at least part of the nose adjacent to and not including the nose bridge and said areas under the eyebrows for a period of time, wherein said skin darkening compound darkens said application area.

16. The method according to claim 15, wherein said mask has an elongated section containing a central slot and two brow protrusions extend from the elongated section that are applied under the eyebrows.

17. The method according to claim 15, wherein said skin darkening compound is a solution of dihydroxyacetone.

18. The method according to claim 17, wherein said solution of dihydroxyacetone is a solution containing between 2 percent and 10 percent dihydroxyacetone.
